(19) United States
(12) Patent Application Publication
(10) Pub. No.: US 2024/0067959 A1
(43) Pub. Date: Feb. 29, 2024

(54) LIBRARY PREPARATION FROM FIXED SAMPLES

(71) Applicant: Saga Diagnostics AB, Lund (SE)

(72) Inventors: Sofia Birkeälv, Furulund (SE); Nuria Segui, Lund (SE); Yilun Chen, Lund (SE); Anthony Miles George, Lund (SE); Lao Hayamizu Saal, Lund (SE)

(21) Appl. No.: 18/240,435

(22) Filed: Aug. 31, 2023

Related U.S. Application Data
(60) Provisional application No. 63/402,511, filed on Aug. 31, 2022.

(51) Int. Cl. C12N 15/10

(52) U.S. Cl. CPC C12N 15/1093

(57) ABSTRACT

Methods of preparing a sequencing library includes fragmenting FFPE-extracted DNA into fragments about 800 bp in length on average; ligating adaptors to the fragments to form adaptor-ligated fragments; size-selecting the adaptor-ligated fragments to provide a mixture enriched for selected adaptor-ligated fragments with a size of about 600 to about 900 bp; and amplifying the selected adaptor-ligated fragments to obtain amplicons.

### TECHNICAL FIELD

The invention relates to extracting DNA from formalin-fixed, paraffin embedded (FFPE) tissue samples and preparing sequencing libraries from the extracted DNA.

### BACKGROUND

Tissue obtained by biopsy or surgery for pathological examination may be fixed in a fixative, such as formalin and embedded in paraffin, yielding formalin fixed, paraffin embedded (FFPE) blocks. Small (5 micrometer-thick) sections may be sliced from the blocks and stained for microscopic analysis. Such slides and the FFPE blocks are typically retained as a pathology archive. It is understood that DNA can be extracted from FFPE blocks. However, it is known that formalin fixation damages DNA. Formaldehyde covalently cross-links DNA, induces oxidation and deamination reactions, and forms derivatives of the four Watson-Crick bases.

Nevertheless, it is desirable that such DNA is extracted and analyzed by sequencing. For example, studies have reported that variant detection can be performed by sequencing FFPE-extracted DNA. Studies have been performed to evaluate different FFPE DNA extraction kits for DNA quality and suitability for variant calling. Such studies have found significant variances among the performance of those kits when variant detection is compared to a baseline gold standard of variant detection such as from fresh-frozen (FF) DNA. Measures of DNA integrity are consistently much lower in FFPE compared to FF samples and the difference is significant. FFPE-extracted DNA is fragmented and typically present only at low molecular weights.

### SUMMARY

The invention provides protocols for extracting DNA from FFPE samples and preparing high-quality sequencing libraries from the FFPE-extracted DNA. The extraction and library preparation protocols are optimized, compared to commercially-available kits and protocols, to compensate for damage that is characteristic of FFPE samples and their extraction. For example, after emulsification of the paraffin, DNA is subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of those steps—limited fragmentation, gentle bead clean-up, and purification after adaptor ligation with size-selection step—may contribute importantly to the preparation of high-quality sequencing libraries from FFPE samples. Compared to prior commercial protocols, other steps may be optimized. For example, after DNA repair and bead clean, high input quantities may be used for adaptor ligation and amplification (e.g., 500 ng instead of 250 ng). In another example, an additional bead clean-up step is added to the protocol after amplification. In another example, the input material may be tested with a quality control assay such as a digital (dPCR) test to qualify the length of fragments. After amplification, another dPCR may be used to quantify yield. In another example, outputs of amplification may be grouped by library yield and groups (based on yield) may be combined for multiplex sequencing. Combining sample first by library yield ensures that sequencing is performed on substantially equimolar library products, which greatly promotes uniform quality of sequencing results.

Combinations of the steps described above promote the extraction of high-quality DNA from FFPE and the preparation of sequencing libraries that will give consistently good results on commercially-available sequencing instruments. Those protocols favor gentle handling and minimal mechanical abuse prior to enzymatic repair and amplification. Enzymatic repair thus cures defects such as oxidative damage that tends to obscure guanine bases in genomic DNA, reading them as thymine bases in sequencing results. Purification and size selection are performed carefully at steps early in these protocols, and additional purification and/or size selection steps may be added after clean-up, repair, ligation, and/or amplification steps. It has been found that protocols according to this disclosure out-perform conventional protocols and kits for extracting and sequencing DNA from FFPE blocks.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline SVs. Such an analysis may provide an identification of tumor-specific somatic SVs actually present in a patient's tumor DNA. That information is then used to design reagents to assay future samples from the patient for those same tumor-specific somatic SVs. In addition, tumor-specific variants discovered using processes of the invention may be useful as generalized markers for structural variants. For example, an informatics pipeline may be used to design amplification primers and fluorescent probes for the detection of such variants by a digital PCR assay. Particular embodiments identify tumor-specific SVs present in a patient's tumor DNA and then use an informatics pipeline to design primers and fluorescent hydrolysis probes useful for detecting by digital PCR those SVs in cell-free tumor DNA in blood or plasma, e.g., from a liquid biopsy.

The ability to monitor for the presence of tumor-specific somatic SVs in a sample from a patient after an initial analysis of a tumor sample, e.g., by creating sequencing libraries from FFPE tumor samples, provides for the detection of the tumor at various times, spanning days, weeks, or years, after an initial biopsy. For example, after treating the patient for cancer, a digital PCR or similar assay using the designed primers and probes may be performed to detect and document an initial impact of the treatment (i.e., whether the treatment is working to reduce tumor burden). In another example, such an assay is performed to detect minimal residual disease (MRD) well after, or at any time after, cancer therapy. An assay, such as digital PCR, for MRD is appealing because it can be minimally invasive and relatively inexpensive, allowing a patient who has been treated for cancer to be tested for MRD regularly after treatment. This provides the ability to detect future disease-recurrence with great sensitivity, i.e., relatively early as compared to conventional methods.

In certain aspects, the invention provides library preparation methods. Exemplary methods include extracting DNA from a formalin-fixed, paraffin embedded (FFPE) tissue sample, fragmenting the DNA into fragments with an average fragment size of at least about 500, preferably at least about 600 or 700, and most preferably at least about 800 base-pairs; and ligating adaptors to the fragments to form adaptor-ligated fragments. A size-selection step is performed to isolate selected adaptor-ligated fragments with an average size within a range from about 500 to about 1000 base-pairs from unwanted material. The selected adaptor-ligated fragments are amplified, e.g., by PCR, to obtain amplicons. The extracting step may include emulsifying paraffin from the tissue sample into a buffer (e.g., by sonication); centrifuging the buffer to form a pellet comprising the DNA; and rehydrating the pellet with lysis buffer (e.g., to liberate DNA from proteins and tissue). The mixture is passed onto a column to capture the DNA from the lysis buffer on the column and the extraction includes eluting the DNA from the column (e.g., using an elution buffer).

In certain embodiments, the fragmenting step involves sonicating eluate from the eluting step. Sonicating may be performed until the eluate reaches an optical density indicating the average fragment size of at least about 500, preferably at least about 600 or 700, and most preferably at least about 800 base-pairs. Optionally, methods may also include using RNA from supernatant from the centrifuging step, e.g., reverse transcribing the RNA and preparing a sequencing library. In some embodiments, methods include\—between the fragmenting and ligating steps—repairing the fragments enzymatically and purifying the repaired fragments, e.g., with magnetic beads at a bead:DNA fragment ratio of less than about 1×, and preferably at about 0.8×. Repairing the fragments may use one or any combination of DNA glycolase, an apurinic/apyrimidinic (AP) endonuclease, DNA polymerase, and ligase. Preferably each of the steps is performed within one or a combination of laboratory test tubes, wells of a plate, microcentrifuge tubes, or tubes in a multi-tube strip. In some embodiments, methods of the invention include performing a bead clean-up on the amplicons, e.g., with a bead:DNA amplicon ratio of less than about 1×, e.g., about 0.8× beads. The method may include measuring a concentration of the amplicons (e.g., with a fluorometer instrument) and/or validating an average size of the amplicons as having an average size with a peak between about 600 and 800 bp (e.g., with an automated electrophoresis instrument).

Methods of the invention may further include sequencing the amplicons to obtain sequence reads; performing a first mapping of the reads to at least one reference by a first algorithm to identify a structural variant; performing a second mapping of the reads by a second algorithm to identify the structural variant; and merging the first mapping with the second mapping to describe the structural variant. For example, the first algorithm may progress by adding the sequence reads to a genomic graph and finding a path through the graph best-supported by the reads; the second algorithm may align read-pairs to a reference and search for genomic regions in the reference where a significant number of read pairs align to the reference in positions anomalous with an empirical insert size distribution for the read pairs. The algorithms may be implemented by software packages such as, for example, GRIDDS and BreakDancer. In some embodiments, methods include sequencing the amplicons to obtain sequence reads; analyzing the sequence reads to identify putative structural variants (SVs) for the DNA; and filtering the putative SVs to remove germline SVs and/or sample handling artefacts, thereby providing a set of somatic SVs present in the DNA. The filtering step may comprise comparing the putative SVs to a database of known germline SVs to remove germline SVs from the putative set of SVs. Methods may include designing, by computer software, at least one primer pair and optionally a probe for each somatic SV in the set, wherein the primer pair will successfully amplify a target that includes the somatic SV. In certain embodiments, methods include using the primer pair to perform an assay from a sample obtained from a subject from whom the FFPE tissue sample was obtained, to detect minimal residual disease in the subject. The assay may be, for example, digital PCR on cell-free DNA from blood or plasma.

In related aspects, the invention provides methods of preparing a sequencing library. Exemplary methods include fragmenting FFPE-extracted DNA into fragments at least about 500, preferably at least about 600 or 700, and most preferably at least about 800 bp in length on average; ligating adaptors to the fragments to form adaptor-ligated fragments; size-selecting the adaptor-ligated fragments to provide a mixture enriched for selected adaptor-ligated fragments with a size of about 600 to about 900 bp; and amplifying the selected adaptor-ligated fragments to obtain amplicons. Methods may include extracting the DNA from a FFPE sample by a process that includes sonicating the sample to emulsify paraffin, centrifuging and re-suspending a resultant in a lysis buffer to liberate DNA from tissue; and purifying the DNA onto a column. Preferably, methods includes purifying, after the fragmenting step and prior to the ligating step, the fragments with magnetic beads at a bead:DNA fragment ratio within a range of about 0.5 to about 0.7; and performing a bead clean-up on the amplicons with a bead:DNA amplicon ratio of about 05 to about 0.7.

### DETAILED DESCRIPTION

The disclosure provides methods of extracting nucleic acids from fixed samples, in which the methods are designed and optimized in view of the fact that fixation and extraction from fixation media otherwise is prone to damage nucleic acid. In an example, it is understood that guanine bases in DNA are prone to oxidation while in FFPE after which a polymerase is liable to incorporate thymine at the guanine position. In another example, available FFPE extraction protocols use acoustic energy, or sonication, to emulsify paraffin and then also use bead clean-up steps. Both of those approaches are mechanical in nature and raise a risk of physical breakage of nucleic acid strands. Those examples illustrate that FFPE storage and extraction may, by their nature, introduce unnatural polymorphisms (e.g., G to T or C to T) and artificial structural variation (breakage) into nucleic acids in a sample.

However, FFPE tissue samples are a common method for storing tumor biopsy specimens. For example, oncologists may want to discover what mutations are specific to a tumor in a patient. Knowledge of such tumor mutations may potentially be used to detect the presence of that tumor in the patient. For example, it is understood that tumors shed cell-free DNA (cfDNA) into the blood of a patient. A blood draw, or liquid biopsy, may be used to sample that circulating tumor DNA (ctDNA). One could potentially analyze ctDNA from a liquid biopsy using knowledge of tumor mutations learned by analyzing FFPE tumor samples. However, existing FFPE storage and extraction protocols introduce polymorphisms and structural variation to nucleic acids. Those variants may be indistinguishable from natural, genetic variation when DNA is sequenced and analyzed. As a result, when nucleic acid from FFPE samples is analyzed for mutations, the results may include both genetic variants, naturally occurring in genetic material, and artifactual variants induced by fixation and extraction protocols.

Methods of the disclosure are useful for extracting DNA from FFPE and minimizing artifactual variants induced by chemical and mechanical insult, while maximizing yield of sequenceable DNA. Compared to existing or known protocols, methods of the invention use mechanical shearing at early stage of the protocols with only minimal levels of energy and only gentle bead clean-steps early at early stages of the protocols, with additional size selection and bead clean-up steps after enzymatic DNA repair. It is noted that preferred paraffin extraction protocols involve emulsifying the paraffin and centrifuging the resultant mixture. At that point, tumor DNA will be in the pellet and supernatant will be enriched for tumor RNA. The pellet can be rehydrated with a lysis buffer (e.g., to liberate the DNA from tissue or cellular material), washed on a column, and eluted from the column. After an initial extraction from paraffin, DNA is only gently sheared, down to a peak length of about 800 to about 1,000 bases compared to 150 bases in conventional protocols. After enzymatic repair and adaptor ligation, an additional size selection step, not found in conventional protocols, is performed, ensuring among other outcomes suitable uniformity among adaptor ligated fragments. Those adaptor-ligated fragments may be amplified (optionally adding indexes or other barcodes for sequencing at any of those stages) to provide a sequencing library, such as a plurality of amplicons with sequencing adaptors at the ends (e.g., Illumina Y-adaptors or similar).

A sequencing library prepared according to methods of the invention from FFPE-extracted DNA from an FFPE tumor sample will contain genetic information of the tumor and can be analyzed to discover tumor-specific mutations. Such library may additionally or alternatively contain amplicons made from cDNA from the RNA from the supernatant from the paraffin extraction step. Approaches to discovering tumor-specific mutations include sequencing, e.g., the tumor DNA sequencing library and analyzing the resultant sequence data to identify tumor mutations including, in particular, structural variants.

Library preparation according to methods of the disclosure preferably begins by extracting DNA from fixed sample. Any fixed sample containing nucleic acid may be used. For example, protocols herein may be used to extract DNA from solid tissue masses, tissue preserved in sap or amber, tissue or nucleic acid preserved in any fixative or fixation medium. Preferred embodiments herein are described with reference to a formalin-fixed, paraffin embedded (FFPE) tissue sample.

A sample may be taken from the FFPE sample, such as a slice or small piece. Steps are performed to extract DNA (and RNA) from that sample. In preferred embodiments, the sample is loaded into a tube such as 0.5 mL screw-cap microcentrifuge tube. A tissue lysis buffer and proteinase K (PK) solution mix may be added to the tube. Such materials may be obtained from a source such as Covaris (Woburn, MA). In fact, many steps of protocols herein may be performed using reagents and material sold under the product name truXTRAC FFPE total NA (tNA) Ultra Kit by Covaris. The FFPE sample is immersed in the tissue lysis buffer/PK solution mix and sonicated in a ultrasonication instrument according to manufacturer instructions for paraffin emulsification. The solution will turn milky white or yellow when emulsifying paraffin from the tissue sample into a buffer by sonication. The tube is preferably then transferred to a heat block and incubated, e.g., for about 30 minutes at about 56 degrees C. Then the tube is briefly cooled.

Each of the steps may be performed in laboratory test tubes, wells of a plate, microcentrifuge tubes, or tubes in a multi-tube strip. The description herein is given in terms of individual microcentrifuge tubes such as the 0.5 mL tube sold as the AFA-TUBE PP Screw-Cap 0.5 mL tube by Covaris. However, one of skill in the art will appreciate that mixtures, emulsification, sonication, centrifuging, column separation, bead clean-up, and other such steps may be performed in tube strips (e.g., a strip of 8 tubes), multi-well plates, traditional (e.g., glass) test tubes, larger (e.g., 50 mL) conical tubes such as those sold under the trademark FALCON by Corning (Corning, NY), or other such containers.

After the tube is cooled, the tube is centrifuged. For example, an 0.5 mL tube may be spun at 5 k g for about 15 minutes. This action will form a pellet that includes DNA and supernatant that may be relatively enriched for RNA. The supernatant is preferably pipetted to a separate tube. At this stage, if it is desired to analyze RNA, the workflow bifurcates, as RNA is analyzed from the supernatant.

For RNA analysis, briefly, the RNA tube is heated (e.g., 80 degree C. for 30 minutes), cooled, treated with a suitable buffer such as Covaris total NA Buffer B1, mixed with isopropanol, and vortexed. Other treatments are suitable and one may extract and isolate RNA by using kits or protocols from commercial vendors. Preferably the reaction mixture is transferred onto an RNA purification column and centrifuged (the column/collection tube assembly are loaded into a microcentrifuge for, e.g., 11 k g for 30 s) with repetitions as necessary until all sample has passed through the column. The column is washed with RNA wash buffer and dried and then treated with an RNA elution buffer. The eluate contains RNA that was in the FFPE tissue sample, which may be referred to as FFPE-extracted RNA. The eluate may be stored on ice or in a freezer until analysis. Any suitable analysis may be performed on the FFPE-extracted RNA.

In some embodiments, the FFPE-extracted RNA is copied into cDNA using a reverse transcriptase and suitable primers. Suitable primers may include gene specific primers (which includes primers designed to anneal to any suitable genetic targets include ribosomal RNA, tRNA, microRNA, mRNA, etc.), poly-T primers to copy from the poly-A tails of mRNA, or random hexamers or similar. First stand synthesis may make use of template-switching oligos (TSOs), which may be used to copy the RNA and a synthetic sequence into the first strand of complementary DNA (cDNA). The synthetic sequence may include a primer binding site for subsequent copying. Second strand synthesis may proceed using nick translational replacement of the mRNA. See Okayama, 1982, High-efficiency cloning of full-length cDNA, Mol Cell Biol 2:161-170 and Gubler, 1983, A simple and very efficient method for generating cDNA libraries, Gene 25:263-269, both incorporated herein by reference. In such embodiments, synthesis of the second strand is catalyzed by E coli DNA polymerase I in combination with E coli RHase H and E coli DNA ligase. The RNase nicks the RNA, providing 3′ hydroxy primers for the DNA polymerase (which has 5′-3′ exo activity) to synthesize segments of the second strand. The ligase links the segments to complete the second strand, forming a dsDNA copy of the RNA. Double stranded cDNA libraries may be created using reagents, kits, and protocols such as the Second Strand cDNA Synthesis Kit from Thermo Fisher Scientific (Waltham, MA). Sequencing adaptors may be ligated to the ds cDNAs, followed by amplification (e.g., PCR) to produce a sequencing library that includes the sequence information of RNA that was in the FFPE tissue sample.

Whether or not it is desired to analyze RNA from the FFPE tissue sample, preferred embodiments of the invention provide protocols for extracting high quality sequenceable DNA with high yield from FFPE tissue samples. After paraffin emulsification, centrifugation produces a pellet that is relatively enriched for the DNA that was in the FFPE tissue sample.

Preferably, the pellet is rehydrated with a suitable buffer such as buffer BE from Covaris and more preferably a tissue lysis buffer/PK solution mix is used. Without being bound by any mechanism, it may be theorized that ultrasonication liberates tissue and cells from paraffin, and that a tissue lysis buffer and/or proteinase (e.g., proteinase K) will aid in liberating DNA from tissue and cellular material, e.g., degrade and hydrolyze cell walls and proteins including DNA binding proteins and chromatin structures. Preferably the pellet is incubated with e.g., about 110 μL buffer BE (Covaris) and about e.g., 400 μL tissue lysis buffer/PK solution mix, mixed (e.g., vortexed), optionally with the tube in an 80 degree heat block.

The tube is sonicated to resuspend material that constitutes the pellet. Sonication instruments will typically include instructions or pre-programmed protocols for pellet resuspension. At this step, the mixture may be stored at room temperatures for e.g., an hour. Also, this is a good step within the workflow to treat the mixture with RNase to remove any residual RNA, if desired. When ready for DNA purification about e.g., 560 μL total NA buffer (Covaris) and about 640 μL 100% ethanol are added. Vortex for about 3 s.

A DNA purification column is placed into a collection tube and one may (i) transfer about 600 μL of sample onto the purification column; (ii) centrifuge the collection tube about 11 k g for about 1 m; and (iii) discard flow-through. Steps (i) through (iii) should be repeated until the entire sample is passed through the column. Following DNA purification protocol instructions, the column is washed with buffer(s) such as BW Buffer and B5 Buffer (Covaris). Finally, the column is eluted with an elution buffer, eluting the DNA from the column. Store eluate containing isolated DNA at 2 degree C. for up to 2 days, or at −20 degree C. for longer term storage.

Methods of the disclosure are provided for producing high quality and high yield sequencing libraries from FFPE-extracted DNA. Having extracted the DNA from the sample by the foregoing steps, methods include fragmenting the DNA.

Methods according to this disclosure include a fragmentation step that is more gentle, less damaging, than existing protocols. Preferably, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. An objective is to produce fragments that have an average size with a peak approximately within the range of about 500, preferably at least about 600 or 700, and most preferably at least about 800 base pairs (bp) to 1,000 bp. Understandably, 500, 600, or 700 bp will work, as will 1,000 bp. A significant point is that current commercial protocols call for shearing to about 150 bp. Here, a cocktail of restriction enzymes may be composed that will, on average, cut genomic DNA on about 800 to 1,000 base intervals. Preferred embodiments use a sonicator or adaptive acoustic focusing (AFA) instrument (Covaris). An important step is to establish the instrument settings for the use case, as samples differ due to storage time. One approach is to use a Qubit instrument to evaluate quantity and/or a TAPESTATION automatic electrophoresis instrument to evaluate fragment length, using manufacturer's literature for guidelines for the sonication instrument, and shear a very small sample to the desired optical density to establish the instrument settings to be used for the bulk of the sample. The instrument is operated only until 800 to 1000 base fragments are achieved, which may be determined by fragmenting test samples to optimize shearing time or by testing the sample being sheared e.g., for optical density or on a gel. Existing, prior protocols may not be expected to work successfully with such long fragments, but other steps of the protocols outlined below have been found to interoperate to consistently yield good results.

The sheared DNA fragments may be analyzed, by way of quality control, prior to library preparation. For example, analysis may be performed using the 2100 Bioanalyzer and DNA 1000 Assay. The Bioanalyzer DNA 1000 chip and reagent kit are used according to manufacturer's instructions to perform the assay according to the Agilent DNA 1000 Kit Guide. The chip, samples and ladder are prepared as instructed in the reagent kit guide, using e.g., 1 μL of sample for the analysis. Load the prepared chip into the instrument and start the run within five minutes after preparation. The electropherogram is inspected to verify a DNA fragment size peak between about 800 and about 1,000 bp. Considering that about means 700 may be suitable and 1,100 may be suitable, possibly even 600 to 1,200, about 800 to about 1,000 bp is the desired size that works in this protocol. Additionally or separately, an automated electrophoresis machine such as those sold under the trademark TAPESTATION by Agilent (Santa Clara, CA) may be used to verify fragment length.

Using, for example, the AFA instrument, the DNA is fragmented into fragments with an average fragment size of at least about 800 base-pairs. In preferred embodiments, after the fragmenting step (but prior to a ligating step below), the DNA is repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA Repair Enzymes and Structure-specific Endonucleases are enzymes which cleave DNA at a specific DNA lesion or structure. Those enzymes can be used for repair of DNA sample degradation due to oxidative damage, UV radiation, ionizing radiation, mechanical shearing, formalin fixation (post extraction) or long term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

Preferably, end-repair is followed by purifying the sample using beads and a magnetic separation device. As stated, this protocol deviates significantly from commercially published protocols (which typically call for a bead:DNA fragment ratio of about 3×). Here, a bead to DNA fragment ratio of about 0.7× is used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. An important feature of this embodiment of the disclosure is the minimal or low-bead ratio, which, in combination with the fragment length and subsequent steps, provides high quality, high-yield sequencing libraries from FFPE samples. Features of this embodiment include that solution above beads is pipetted away, and ethanol is added to wash the sample (which can be repeated). Then, the sample may be subjected to spin to collect at the bottom and subjected to air drying to remove excess ethanol and evaporate residual ethanol in the thermocycler. Nuclease-free water may be pipetted into the tube, which dissolves or resuspends the DNA off of the beads. The resulting solution is vortexed briefly and exposed to a magnet for e.g., about 2 or 3 minutes. The clear supernatant that includes the end-repaired, FFPE-extracted DNA fragments is then removed and the beads are discarded. Other embodiments do not need a full wash. In such embodiments, after removal of restriction enzymes, DNA is eluted into the ligation mix, and then the ligation is performed with the beads in solution, since there is no PEG/NaCl the DNA is in solution. In such embodiments, after ligation, reaction enzymes are cleaned away by adding PEG/NaCl e.g. DNA binds back to the beads.

In any embodiment, the above protocols include ligating adaptors to the fragments to form adaptor-ligated fragments. Any suitable approach may be used. Some embodiments include dA tailing the 3′ end of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). That kit includes reagents and instructions for a Ligation 1 in which a Ligation 1 Enzyme catalyzes the single-stranded addition of the Ligation 1 Adapter to only the 3′ end of the insert. That enzyme is unable to ligate inserts together, which minimizes the formation of chimeras, which in turn improves the false-positive rates for fusions. The 3′ end of the Ligation 1 Adapter also contains a blocking group to prevent adapter-dimer formation. Then, a Ligation 2 Adapter acts as a primer to gap-fill the bases complementary to the Ligation 1 Adapter, followed by ligation to the 5′ end of the DNA insert to create a double-stranded product. That double-stranded adaptor ligated product is suitable for amplification by PCR using indexing primers. However, this protocol according to this invention does not proceed straight to PCR at this point. Instead, a size selection step is performed first.

Preferably, the adaptor ligated fragments are subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp. Any suitable approach to size selection may be used, including gel electrophoresis and band excision, size exclusion chromatography, bead purification with controlled bead: DNA ratios, or other methods. It will be understood that beads can be used for simultaneous clean-up & size selection by manipulating the ratio of bead buffer (PEG+salt) volume to sample volume. Lower bead buffer to sample volume ratios correlate with larger sizes retained, and thus smaller sized materials such as primers and adaptors are removed in the clean-up.

One suitable approach for the tight size-selection to about 550 to 900 bp includes: vortexing AMPure XP beads to resuspend them; adjusting the final volume after ligation by adding nuclease free water; adding resuspended AMPure XP beads to the ligation reaction at [A] a first bead ratio; followed by mixing: incubating for 5 minutes at room temperature; spinning; placing on a magnetic stand to separate the beads from the supernatant; transferring the supernatant containing the DNA to a new tube; and adding resuspended AMPure XP beads to the supernatant at [B] a second bead ratio; mixing well and incubating for 5 minutes at room temperature; spinning; placing on a magnetic stand to separate the beads from the supernatant; once clear removing and discarding the supernatant—beads contain the desired DNA targets; adding ethanol and discarding supernatant to wash; repeating the wash; air drying beads; eluting the DNA target from the beads into Tris-HCl or TE; mixing; spinning; placing on a magnetic stand; and once clear, transferring solution to a new PCR tube for amplification. The foregoing short description gives a general purpose approach to size selection by bead purification. A fragment size can be selected for by careful choice of the “[A] first bead ratio” and “[B] second bead ratio”.

The selected adaptor-ligated fragments should have an average size within a range of about 500 to about 1000 bp, specifically preferably within the range of 550-900 bp. A fragment size within a range of about 550 to 900 bp may be obtained by using about 0.30 and 0.15 for the [A] first bead ratio and [B] second bead ratio. Those values may vary based on the particular FFPE tissue sample being used (time of storage, chemical nature of fixatives, DNA abundance in original tumor, etc.) so a suitable step may be to perform optimization reactions on very small portions of the solution and validate the results on a TAPESTATION instrument to determine the bead ratios and other conditions for the tight size selection step after adaptor ligation and prior to PCR.

The selected adaptor-ligated fragments are amplified to obtain amplicons. PCR reaction volumes should be adjusted to accept all material obtained from the tight size selection step. Here, commercial instructions provide that a maximum amount of input material is 250 ng, but this protocol finds benefit from using higher amounts, even up to about 500 ng.

In one embodiment, the adaptors preferably include barcodes. Those barcodes may include sample barcodes, unique molecular identifiers (UMIs), other barcodes, and any combination thereof. As noted above, of the invention comprises obtaining RNA from supernatant after emulsifying paraffin. The use of UMIs may benefit any application or use of the invention and may find particular benefit where RNA and DNA are made into sequencing libraries.

A unique molecular identifier is generally a barcode sequence that functions as if it were unique and is attached to genetic material (DNA or RNA) to be sequenced. Interestingly, UMIs need not be truly unique and are sometimes described as “unique or nearly unique”. Because nucleic acid molecules are amplified prior to sequencing and, in many platforms, essentially amplified again as part of the sequencing protocol, the abundance of data that result from sequencing does not reflect, necessarily, an amount or number of input nucleic acids. Sequencing produces sequence reads. In many platforms, sequencing produces short sequence reads, e.g., between about 35 and 50 bases in length of data from the nucleic acid from the sample. If two of those reads are identical (e.g., duplicates), one may not otherwise know if they originate from two different molecules in the sample or from clonal copies of one original molecule made during amplification. By tagging each original molecule with a UMI, sequence reads will (essentially) only be duplicates if they originated from the same molecule of nucleic acid that was present in the sample. After sequencing, software may be used to de-duplicate sequence reads (sometimes referred to as collapsing reads), leaving only one sequence read per molecule from the sample. If UMIs are used and sequence reads are de-duplicated, then a count of unique sequence reads is a measure of molecules in a sample. In one example, if a cell in an FFPE sample had been expressing genes named yfg1 and yfg2, the cell may have millions of copies of yfg1 mRNA and only hundreds of copies of yfg2 mRNA. Sequencing the RNA from that sample using UMIs as described will reveal the relative expression levels of those genes, which may have biological importance.

After size selection, the selected fragments are amplified by PCR. In this embodiment, PCR reaction volumes are preferably adjusted to accept all material obtained from the tight size selection step. Here, commercial instructions provide that a maximum amount of input material is 250 ng, but methods of the invention benefit from using higher amounts, even up to about 500 ng. In most cases, it will be suitable to amplify only a portion of the fragments (the PCR input), and the remainder may be kept in a freezer. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage.

After PCR, some conventional protocols describe a bead cleanup step. See, for example, Agilent, 2021, SureSelectXT Target enrichment system for the Illumina Platform, Protocol, Agilent Technologies (102 pages), incorporated by reference, which at Step 11 describes purifying an amplified library with a 90:50 bead:DNA ratio. In the present disclosure, to maximize library yield and quality for sequencing libraries prepared from FFPE-extracted DNA, such a bead cleanup is preferably performed on the amplicons with a bead:DNA amplicon ratio of less than about 1, most preferably the ratio is about 0.8.

At this stage, a library preparation is complete, except that numerous samples may be run separately (e.g., in parallel) and this protocol provides guidance for handling multiple libraries for best results when sequencing. As an initial matter, any given library may be subject to quality control steps. Checking the quality of a sequencing library may involve looking at any relevant feature of the library. Relevant features may include quantity and/or amplicon size. The quantity of DNA in a sequencing library may be determined using a fluorometer such as the fluorometer sold under the trademark QUBIT by Thermo Fisher Scientific. Amplicon sizes may be measured using an automatic electrophoresis tools such as the TAPESTATION-branded instrument from Agilent. Additionally or alternatively, library yield may be quantified by digital PCR. Such steps may be performed for measuring a concentration of the amplicons and/or validating an average size of the amplicons as having an average size with a peak between about 600 and 800 bp.

When multiple libraries (e.g., from different tumor slices in paraffin) are prepared, while the tubes may look similar, there may be diversity in contents, in terms of library yield. It has been found that sequencing results may be optimized by dividing libraries into a different sequencing pools according to their determined yields, and then combining libraries equimolarly according to their quantities. Absent this step, without being bound by any mechanism, it may be theorized that different libraries present highly different amounts of starting material onto an Illumina flow cell, and the abundant library may simply rapidly outpace other during bridge amplification, usurp reagents, or dominate the instrument read capability.

The present disclosure comprises protocols for creating high-yield, high-quality sequencing libraries from FFPE-tissue samples. Those libraries may be stored or held in any suitable container or format and/or used in any suitable assay or experiment. For example, sequencing libraries according to the invention may placed in a tube such as an 0.5 mL microcentrifuge tube and stored in a freezer at a suitable temperature, such as −20 degrees C. In another example, a suitable handling of a sequencing library according to the present invention includes placing the amplicons in a tube, placing the tube on dry ice in a Styrofoam (or similar) shipping container, and shipping the container to a genomics core facility or other such facility to have the amplicons sequenced. In certain embodiments of the disclosure, the described methods include sequencing the amplicons to obtain sequence reads. Sequencing produces a plurality of sequence reads that may be analyzed to detect structural variants. Sequence read data can be stored in any suitable file format including, for example, VCF files, FASTA files or FASTQ files, as are known to those of skill in the art. In some embodiments, PCR product is pooled and sequenced (e.g., on a sequencing instrument such as an Illumina HiSeq 2000). Raw .bcl files are converted to qseq files using bclConverter (Illumina) or to fastq files using bcl2fastq (Illumina). FASTQ files are generated by “de-barcoding” genomic reads using the associated barcode reads; reads for which barcodes yield no exact match to an expected barcode, or contain one or more low-quality base calls, may be discarded. Reads may be stored in any suitable format such as, for example, FASTA or FASTQ format.

FASTA is originally a computer program for searching sequence databases and the name FASTA has come to also refer to a standard file format. See Pearson & Lipman, 1988, Improved tools for biological sequence comparison, PNAS 85:2444-2448, incorporated by reference. A sequence in FASTA format begins with a single-line description, followed by lines of sequence data. The description line is distinguished from the sequence data by a greater-than (“>”) symbol in the first column. The word following the “>” symbol is the identifier of the sequence, and the rest of the line is the description (both are optional). There should be no space between the “>” and the first letter of the identifier. It is recommended that all lines of text be shorter than 80 characters. The sequence ends if another line starting with a “>” appears; this indicates the start of another sequence.

The FASTQ format is a text-based format for storing both a biological sequence (usually nucleotide sequence) and its corresponding quality scores. It is similar to the FASTA format but with quality scores following the sequence data. Both the sequence letter and quality score are encoded with a single ASCII character for brevity. The FASTQ format is a de facto standard for storing the output of high throughput sequencing instruments such as the Illumina Genome Analyzer. Cock et al., 2009, The Sanger FASTQ file format for sequences with quality scores, and the Solexa/Illumina FASTQ variants, Nucleic Acids Res 38(6):1767-1771, incorporated by reference.

For FASTA and FASTQ files, meta information includes the description line and not the lines of sequence data. In some embodiments, for FASTQ files, the meta information includes the quality scores. For FASTA and FASTQ files, the sequence data begins after the description line and is present typically using some subset of IUPAC ambiguity codes optionally with “−”. In a preferred embodiment, the sequence data will use the A, T, C, G, and N characters, optionally including “−” or U as-needed (e.g., to represent gaps or uracil, respectively).

Following sequencing, reads may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated herein by reference.

Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303, incorporated by reference (aka the GATK program). Reads may be assembled using SSAKE version 3.7. The resulting contiguous sequences (contigs) can be aligned to the reference (e.g., using BWA).

In some embodiments, a sequence alignment is produced-such as, for example, a sequence alignment map (SAM) or binary alignment map (BAM) file-comprising a CIGAR string (the SAM format is described, e.g., in Li, et al., The Sequence Alignment/Map format and SAMtools, Bioinformatics, 2009, 25(16):2078-9, incorporated by reference). Output from mapping may be stored in a SAM or BAM file, in a variant call format (VCF) file, or other format. In an illustrative embodiment, output is stored in a VCF file. A typical VCF file will include a header section and a data section. The header contains an arbitrary number of meta-information lines, each starting with characters ‘##’, and a TAB delimited field definition line starting with a single ‘#’ character. The VCF format is described in Danecek et al., 2011, The variant call format and VCFtools, Bioinformatics 27(15):2156-2158, incorporated by reference.

Regardless of small variants (polymorphisms and small indels) that may be found by mapping the sequence data, methods of the invention preferably analyze the read to detect tumor-specific somatic structural variants. Preferred embodiments employ a computational pipeline that uses two different algorithms, each intended for finding SVs, to call putative SVs and merge the results. The computation pipeline is used for a method that includes performing a first mapping of the reads to at least one reference by a first algorithm to identify a structural variant; performing a second mapping of the reads by a second algorithm to identify the second structural variant; and merging the first mapping with the second mapping to describe the structural variant. In preferred embodiments, the first algorithm adds the reads to a genomic graph and finds a path through the graph best-supported by the reads. This approach may be implemented by a suitable software platform such as GRIDSS. Methods may include software, tools, and techniques described in Cameron, 2017, GRIDSS: sensitive and specific genomic rearrangement detection using positional de Bruign graph assembly, Genome Research 27(12):2050-2060 and Cameron, 2021, GRIDSS2: comprehensive characterization of somatic structural variation using single breakend variants structural variant phasing, Genome Biol 22(1):202, both incorporated by reference. Preferably, the second algorithm aligns read-pairs to a reference and searches for genomic regions in the reference where a significant number of read pairs align to the reference in positions anomalous with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9):677-681, incorporated by reference.

Using such tools, the methods include sequencing the amplicons to obtain sequence reads; analyzing the sequence reads to identify putative structural variants (SVs) for the DNA; and then filtering the putative SVs to remove germline SVs and/or sample handling artefacts, thereby providing a set of somatic SVs present in the DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing FFPE-extracted DNA, a variant calling pipeline may find those benign germline variants. Typically, one is more interested in somatic mutations that are specific to a tumor (from which the FFPE sample was created). Thus, all SVs found by sequencing are preferably filtered to remove benign germline variants from the putative set, leaving a set of tumor-specific somatic SVs.

Once that set is determined, methods may include designing, by computer software, at least one primer pair for each somatic SV in the set, wherein the primer pair will successfully amplify a target that includes the somatic SV. That primer pair may be used to perform an assay from a sample from a subject from whom the FFPE tissue sample was obtained, to detect minimal residual disease in the subject. In preferred embodiments, that assay involves digital PCR on cell-free DNA from blood or plasma, or a “liquid biopsy”.

As described, the disclosure provides protocols for preparing a sequencing library. Such methods include fragmenting FFPE-extracted DNA into fragments at least about 800 bp in length on average; ligating adaptors to the fragments to form adaptor-ligated fragments; size-selecting the adaptor-ligated fragments to provide a mixture enriched for selected adaptor-ligated fragments with a size of about 600 to about 900 bp; and amplifying the selected adaptor-ligated fragments to obtain amplicons. The DNA may be extracted from a FFPE sample by a process that includes sonicating the sample to emulsify paraffin, centrifuging and re-suspending a resultant in a lysis buffer to liberate DNA from tissue; and purifying the DNA onto a column. Methods may include purifying, after the fragmenting step and prior to the ligating step, the fragments with magnetic beads at a bead:DNA fragment ratio in a range of about 0.5 to about 0.7; and performing a bead clean-up on the amplicons with a bead:DNA amplicon ratio in a range of about 0.5 to about 0.7.

What is claimed is:

1. A library preparation method comprising:
extracting DNA from a formalin-fixed, paraffin embedded (FFPE) tissue sample;
fragmenting the DNA into fragments with an average fragment size of at least about 500 base-pairs;
ligating adaptors to the fragments to form adaptor-ligated fragments;
isolating selected adaptor-ligated fragments with an average size within a range of about 500 to about 1000 base-pairs from unwanted material; and
amplifying the selected adaptor-ligated fragments to obtain amplicons.

2. The method of claim 1, wherein the average fragment size is at least about 700 base-pairs, preferably at least about 800 base-pairs.

3. The method of claim 1, wherein the extracting step comprises:
emulsifying paraffin from the tissue sample into a buffer;
centrifuging the buffer to form a pellet comprising the DNA;
rehydrating the pellet with lysis buffer;
capturing the DNA from the lysis buffer onto a column; and
eluting the DNA from the column.

4. The method of claim 3, wherein the fragmenting step comprises sonicating an eluate from the eluting step.

5. The method of claim 4, wherein the sonicating is performed until the eluate reaches an optical density indicating the average fragment size of at least about 800 base-pairs.

6. The method of claim 3, further comprising reverse transcribing RNA from a supernatant from the centrifuging step.

7. The method of claim 1, further comprising, after the fragmenting step and prior to the ligating step:
repairing the fragments enzymatically; and
purifying the fragments with magnetic beads at a bead:DNA fragment ratio of less than about 1.

8. The method of claim 7, wherein the repairing step is performed using one or a combination of a DNA glycolase, an apurinic/apyrimidinic (AP) endonuclease, a DNA polymerase, and a ligase.

9. The method of claim 1, wherein each of the steps is performed within one or a combination of laboratory test tubes, wells of a plate, microcentrifuge tubes, or tubes in a multi-tube strip.

10. The method of claim 1, further comprising performing a bead clean-up on the amplicons with a bead:DNA amplicon ratio of less than about 1.

11. The method of claim 1, further comprising
measuring a concentration of the amplicons; and/or
validating an average size of the amplicons as having an average size with a peak between about 600 and 800 bp.

12. The method of claim 1, further comprising sequencing the amplicons to obtain sequence reads; performing a first mapping of the reads to at least one reference by a first algorithm to identify a structural variant; performing a second mapping of the reads by a second algorithm to identify the structural variant; and merging the first mapping with the second mapping to describe the structural variant.

13. The method of claim 12, wherein the first algorithm adds the reads to a genomic graph and finds a path through the graph best-supported by the reads and wherein the second algorithm aligns read-pairs to a reference and searches for genomic regions in the reference where a significant number of read pairs align to the reference in positions anomalous with an empirical insert size distribution for the read pairs.

14. The method of claim 1, further comprising sequencing the amplicons to obtain sequence reads; analyzing the sequence reads to identify putative structural variants (SVs) for the DNA; and filtering the putative SVs to remove germline SVs and/or sample handling artefacts, thereby providing a set of somatic SVs present in the DNA.

15. The method of claim 14, wherein the filtering step compares the putative SVs to at least one database of known germline SVs and removing matched germline SVs from the putative SVs.

16. The method of claim 14, further comprising designing, by computer software, at least one primer pair for each somatic SV in the set, wherein the primer pair will successfully amplify a target that includes the somatic SV.

17. The method of claim 16, further comprising using the primer pair to perform an assay from a sample from a subject from whom the FFPE tissue sample was obtained, to detect minimal residual disease in the subject.

189. The method of claim 17, wherein the assay comprises digital PCR on cell-free DNA from blood or plasma.

19. A method of preparing a sequencing library, the method comprising:
fragmenting FFPE-extracted DNA into fragments at least about 800 bp in length on average;
ligating adaptors to the fragments to form adaptor-ligated fragments;
size-selecting the adaptor-ligated fragments to provide a mixture enriched for selected adaptor-ligated fragments with a size of about 600 to about 900 bp; and
amplifying the selected adaptor-ligated fragments to obtain amplicons.

20. The method of claim 19, further comprising the FFPE-extracted DNA from a FFPE sample by a process that includes sonicating the sample to emulsify paraffin, centrifuging and re-suspending a resultant in a lysis buffer to liberate DNA from tissue; and purifying the DNA onto a column.

21. The method of claim 19, further comprising:
purifying, after the fragmenting step and prior to the ligating step, the fragments with magnetic beads at a bead:DNA fragment ratio in range of about 0.5 to about 0.7; and
performing a clean-up on the amplicons with a bead:DNA amplicon ratio in a range of about 0.5 to about 0.7.
